(19) United States
(12) Patent Application Publication
(10) Pub. No.: US 2010/0180897 A1
(43) Pub. Date: Jul. 22, 2010

(54) COUGHING AID DEVICE

(76) Inventor: Michel Malgouyres, Montpellier (FR)

Correspondence Address: JEROME D. JACKSON (JACKSON PATENT LAW OFFICE), 211 N. UNION STREET, SUITE 100, ALEXANDRIA, VA, 22314, US

(21) Appl. No.: 12/664,402

(22) PCT Filed: Jun. 27, 2008
(86) PCT No.: PCT/FR2008/000922

§ 371 Date: Mar. 5, 2010

(30) Foreign Application Priority Data
Jun. 28, 2007 (FR) 0704657

(51) Int. Cl. A61M 16/00

(52) U.S. Cl. 128/204.23; 128/204.21

(57) ABSTRACT

The invention relates to a coughing aid device, comprising a first-sub-assembly (1) with a ventilation group (11), an air control unit (12) and an electronic management device (13) and a second sub-assembly (2) with a mask (21), an antibacterial filter (22) and a distribution tube (23) connected to the first assembly (1). Said device is essentially characterised in that the electronic device (13) is made up of a) a storage memory for preset programmes corresponding to expectoration types by pathology type and patient profile, b) a microcontroller operating the ventilation group (11) and the air control unit (12), by means of the preset programmes contained in said storage memory, a flow sensor (16) and a pressure sensor (17) which measure the flow and pressure in real time in order to compare the same with prerecorded flow, pressure and inhaled and exhaled volume limit values and to act immediately on the means for controlling the ventilation group (11) and the air control regulation unit (12).

### FIELD OF THE INVENTION

The present invention relates to a coughing aid apparatus facilitating bronchial and pulmonary decongestion by improving the expectoration of mucus by external assistance.

### TECHNOLOGICAL BACKGROUND

The known apparatuses of the kind in question generally comprise:

a) a first sub-assembly comprising:

a ventilation unit provided with a turbine and an air control unit provided with means enabling connection of the aforementioned ventilation unit with the patient in inflow as well as outflow and a means, of the regulation solenoid valve type, adapted to produce series of pulsations of air, positive pulsations during the inspiration phase and negative pulsations during the expiration phase;

an electronic unit adapted to control the aforementioned ventilation unit and the aforementioned air control unit;

b) a second subassembly comprising a mask, an antibacterial filter and a distribution tube connected to the first assembly.

Let us keep in mind that respiration is divided into 4 successive phases which are:

1—the inspiration phase where the inspiration volume increases;

2—the inspiratory pause phase where the flow rate is null;

3—the expiration phase where the flow rate shows a peak at the first moment to become moderated until the end of the aforementioned phase;

4—the expiratory pause phase where the flow rate is null; and that it is at the time of the 1st phase that the intra alveolar pressure is greater than the intra thoracic pressure, within the chest with the consequences:

the opening of the alveoli allowing an evacuation of mucus;

an effective expectoration due to the extent and the early occurrence of peak expiratory flow rate.

The closest prior art is described in document WO 2005/025658.

The apparatus in question does not comprise means of the microcontroller types and associated memory enabling storage and modification of preset programs corresponding to expectoration type profiles by type of disease and patient profile.

It also does not comprise means enabling:

effectuation of measurements, in real time, of flow rate and of pressure in order to compare to prerecorded limit values for volume, inspiratory and expiratory pressure and flow rate and immediate action on the air regulation solenoid valve;

communication with a remote station;

action by remote control by the patient on certain system functions.

### SUMMARY OF THE INVENTION

The present invention is intended to implement a system that has the following therapeutic advantages:

the storage of settings, in order to carry on the sessions in the best conditions;

the control of maximum inspiration parameters such as the volume, the flow rate and the pressure in order to limit the risk of pneumothorax;

the control of maximum expiration parameters such as the volume, the flow rate and the pressure as well as the maximum cough flow rate to limit the risk of collapse;

the prevention of a desaturation via the (optional) use of an oximeter that can stop the cycle if necessary;

the oscillation of the expiratory pressure enabling fluidification of mucus (the thixotropy phenomenon);

monitoring of the treatment at home.

To this end, an object of the invention is a coughing aid apparatus that is characterized essentially in that the electronic unit is constituted:

a) by a storage memory for preset programs corresponding to expectoration type profiles by type of disease and patient profile;

b) by a microcontroller that operates the ventilation unit and the air control unit:

first, by means of preset programs contained in the aforementioned storage memory, so as to generate a pressure, positive pressure during the inspiration phase and negative pressure during the expiration phase, whose values of flow rate and pressure and the number and duration of inspiration and expiration cycles are configurable;

second, by means of a flow rate sensor and a pressure sensor that measure, in real time, the flow rate and the pressure, measurements that are compared to prerecorded limit values for inspiratory and expiratory volumes, pressure, and flow rate, in order to act immediately on the velocity of the turbine of the ventilation unit and on the means for regulation of the air control unit.

The apparatus also comprises:

an oximeter adapted to provide to the microcontroller the heart rate and rate of saturation of hemoglobin in the blood (SpO2) of the patient;

a human/machine interface provided, in particular, with a LCD screen, a set of buttons and a remote analog scroll wheel adapted to enable the setting and the use of the apparatus in connection with the microcontroller.

### PRESENTATION OF FIGURES

The characteristics and the advantages of the invention will appear more clearly upon reading the detailed description that follows of at least one preferred implementation mode thereof given by way of nonlimiting example and shown in the accompanying drawings.

In the drawings:

FIG. 1 is a schematic view of the assembly according to the invention;

FIG. 2 is a graph illustrating pressure as a function of time and showing the positive and negative pulsations of air during a cycle of inspiration and expiration as well as their possible modulation.

### DETAILED DESCRIPTION OF THE INVENTION

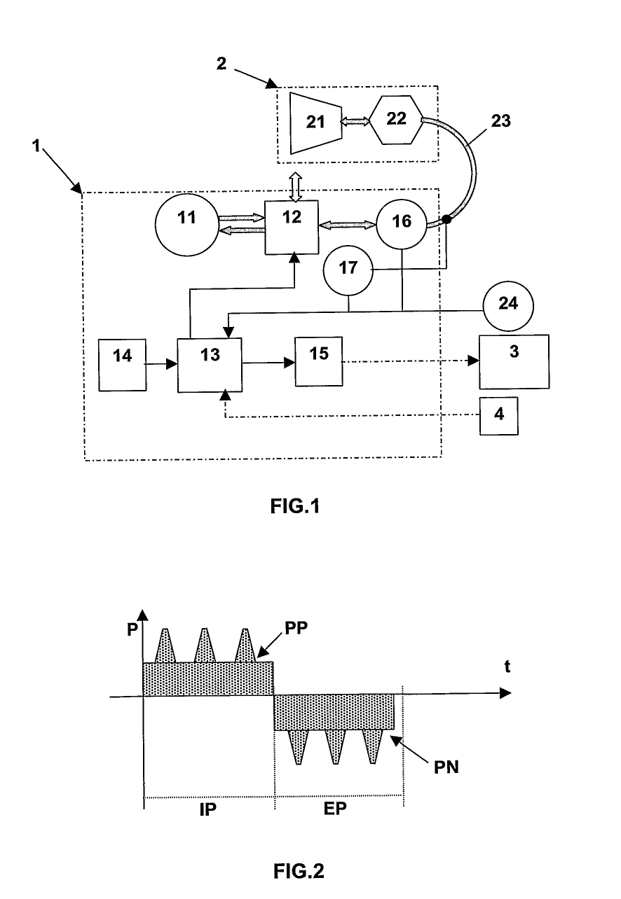

The assembly shown in FIG. 1 comprises:

a) a first subassembly (1) comprising:

a ventilation unit (11) provided with a turbine that creates a flow rate of air, thus an induced pressure, and an air control unit (12) provided with means enabling connection of the aforementioned ventilation unit with the patient, in inflow as well as outflow (as well as with the atmosphere and with an oxygen source) and a means, of the regulation solenoid valve type, adapted to produce series of pulsations of air, positive pulsations (PP) during the inspiration phase and negative pulsations (PN) during the expiration phase;

an electronic unit (13) adapted to control the aforementioned ventilation unit and the aforementioned air control unit;

b) a second subassembly (2) comprising a mask (21), an antibacterial filter (22) and a distribution tube (23) connected to the first assembly (1).

The diagram shown in FIG. 2 illustrates the series of pulsations of air, positive (PP) during the inspiration phase (IP) and negative (PN) during the expiration phase (EP) to which the patient is subjected. The corresponding pulsations are programmable in frequency and pressure and can be modulated by an AC component.

The electronic unit (13), is constituted:

a) by a storage memory for preset programs corresponding to expectoration type profiles by type of disease and patient profile;

b) by a microcontroller that operates the ventilation unit (11) and air control unit (12):

first, by means of preset programs contained in the aforementioned storage memory, so as to generate a pressure, positive during the inspiration phase (IP) and negative during the expiration phase (EP), whose values of flow rate and pressure and the number and duration of inspiration and expiration cycles are configurable;

second, by means of a flow rate sensor (16) and a pressure sensor (17) that measure, in real time, the flow rate and the pressure in order to compare them to prerecorded limit values for inspiratory and expiratory volumes, pressure, and flow rate and act immediately on the velocity of the turbine of the ventilation unit (11) and on the means for regulation of the air control unit (12).

The positive (PP) and negative (PN) air pulsations are modulated by an AC component.

The measurement of the pressure upstream from the bacterial filter is taken into account to calculate the pressure at the mask/patient interface.

The first assembly (1) also comprises:

a communication interface (15) designed to transfer, to a remote station (3), data stored in the memory, the aforementioned interface being designed for receiving means selected among: a proprietary interface, a specific USB key enabling transfer and retention of the medical data of the patient, an Internet connection or a modem (wired or not), a GSM connection;

alarms for voltage limits of the power source, pressures and the temperature. According to other implementation possibilities of the invention:

a remote interface (4), with or without wire, employable by the patient, can enable for example the modulation of the velocity of the turbine, the commanded changeover from the drawn pressure regime to the continuous pressure regime, from the positive pulsations regime to the negative pulsations regime, and vice versa. This remote control interface comprises at least a command means, scroll wheel or joystick, for example, and a means of connection with the first sub-assembly (1).

the distribution of air can be complemented by the distribution of oxygen in the case of an oxygen-dependent patient;

a “pressure relaxer” program can be incorporated into the apparatus. The microcontroller enables:

the setting/use of the apparatus by means of a human/machine interface (14) comprising a LCD screen, a set of buttons and a remote analog scroll wheel (4) provided with information on its position;

the control of velocity of the turbine by means of a power interface;

the selection of the direction and the modulation of the flow rate via the control unit (12);

the storage of settings associated with various user profiles in embedded flash memory

the storage and the timestamp of user sessions via an embedded real time clock;

the communication of data stored in a third party electronic device of the “loader” type or a PC;

the use of PC the creation, the retrieval and the modification of profiles via a dedicated program;

the calculation of volumes of air entering and exiting by integration of the measured flow rate;

the taking into account and display of the oximetry parameters of the patient by means of an optional oximeter (24): heart rate and rate of saturation of hemoglobin in the blood (SpO2).

Several operating modes are possible:

1) Automatic: it controls the durations of inspiration, expiration, intra cycle and inter cycle respiratory pause, predefined via the human/machine interface (HMI);

2) Manual: the succession of cycles and the dynamics of flows of airs are controlled by the HMI in real time (via an analog scroll wheel);

3) Semi Automatic: the transition from one cycle to another is effectuated according to the analysis of predefined parameters. (Respiratory activity, inspiratory and expiratory pressures and volumes).

The operating principle is the following:

1) The inspiration

1-1. The trigger: it is initialized by the detection of an inspiration, or via the HMI (scroll wheel) or after a delay following the expiratory pause, or a duration defined via the HMI;

1-2. The controllable parameters (definable via the HMI):

The maximum inspiration pressure: the microcontroller controls the ventilation unit and the air control unit so that the pressure is not greater than the maximum pressure (reduction of risks of pneumothorax);

the maximum volume of inspiration: the volume calculated from the flow rate meter (16) and from the microcontroller stops the flow when the volume is equal to the maximum inspiration volume (reduction of risks of pneumothorax);

the maximum inspiratory flow;

the angular velocity of the turbine: it can be controlled by HMI (scroll wheel) while adhering to the maximum values of inspiration pressure, volume, and flow rate: the velocity of the turbine can be managed as a function of the feeling of the patient because the patient has an impact on the flow rate and the pressure;

the duration of inspiration in the case of automated cycles.

1-3. The end of the inspiration cycle: it can be timed or triggered when the maximum pressure is attained, the measured flow rate is sufficiently low, the maximum inspiratory volume is attained by the HMI (scroll wheel) or by the detection of an expiration force.

2) The inspiratory pause

It starts at the end of the inspiration and ends after a delay regulated via the HMI or after the detection of an expiration or via the HMI (analog scroll wheel).

3) The expiration

3-1. The trigger: it starts at the end of the inspiratory pause.

3-2. The possible settings:

the percussions: via the control unit (12) the flow-rate/pressure pair created by the unit (11) can be modulated: The settings enable setting of the modulation frequency (Hz) as well as the amplitude (absolute value or % of the current pressure);

the peak expiratory flow rate (PCEF) and its duration (occur at the start of expiration)

the maximum expiratory volume

the maximum expiratory pressure

the expiratory flow rate after the PCEF: it can be controlled by the analog scroll wheel; it can define a post PCEF value of maximum expiratory flow rate;

the angular velocity of the turbine: it can be controlled by the HMI (scroll wheel) while adhering to the maximum values of expiratory pressure, volume, and flow rate: the velocity of the turbine can be managed as a function of the feeling of the patient because the patient has an impact on the flow rate and the pressure that depends on the patient's activity.

3-3. The end of the expiration cycle: it can be timed or triggered via the HMI (scroll wheel), after the detection of an end of expiration or when the maximum expiration volume is attained.

3-4. The expiratory pause: it starts at the end of expiration and ends at the start of inspiration.

An operation mode and various parameters settings by a user can be associated; this entry is made via the HMI or at the time of connection of the DM to a PC via a profile management program.

The uses are stored: conditions, timestamp, etc.

The recovery of data storage via a “loader” or directly with a PC enables a monitoring of the execution of the procedure (especially useful for an apparatus provided during a home hospitalization).

The machine has preset profiles adapted to various types (infant, child, adult, . . . ).

Of course, the person of skill in the art will be capable of implementing the invention as described and shown, applying and adapting known means.

He will also be able to foresee other variations without consequently departing from the scope of the invention that is determined by the content of the claims.

1. Coughing aid apparatus comprising:
a) a first subassembly comprising:
a ventilation unit provided with a turbine and an air control unit provided with means enabling connection of the aforementioned ventilation unit with the patient in inflow as well as outflow and a means, of the regulation solenoid valve type, adapted to produce series of pulsations of air, positive pulsations during the inspiration phase and negative pulsations during the expiration phase;
an electronic unit adapted to control the aforementioned ventilation unit and the aforementioned air control unit;
b) a second subassembly comprising a mask, an antibacterial filter and a distribution tube connected to the first assembly; characterized in that the electronic unit, is constituted:
a) by a storage memory for preset programs corresponding to expectoration type profiles by type of disease and patient profile;
b) by a microcontroller that operates the ventilation unit and the air control unit:
first, by means of preset programs contained in the aforementioned storage memory, so as to generate a pressure, positive during the inspiration phase (IP) and negative during the expiration phase (EP), whose values of flow rate and pressure and the number and duration of inspiration and expiration cycles are configurable;
second, by means of a flow rate sensor and a pressure sensor that measure, in real time, the flow rate and the pressure in order to compare them to prerecorded limit values for inspiratory and expiratory volumes, pressure, and flow rate and act immediately on the velocity of the turbine of the ventilation unit and on the means for regulation of the air control unit.

2. Apparatus, according to claim 1, characterized in that the positive and negative air pulsations are modulated by an AC component.

3. Apparatus, according to claim 1, characterized in that it comprises an oximeter adapted to provide to the microcontroller the heart rate and rate of saturation of hemoglobin in the blood of the patient.

4. Apparatus, according to claim 1, characterized in that it comprises a human/machine interface provided, in particular, with a LCD screen, a set of buttons and an analog scroll wheel adapted to enable the setting and the use of the apparatus in connection with the microcontroller.

5. Apparatus, according to claim 1, characterized in that the first sub-assembly comprises a communication interface designed to transfer, to a remote station, data stored in the memory.

6. Apparatus, according to claim 5, characterized in that the communication interface is designed for receiving means selected among: a proprietary interface, a specific USB key enabling transfer and retention of the medical data of the patient, an Internet connection or a modem, a GSM connection.

7. Apparatus, according to claim 1, characterized in that it comprises a remote interface, with or without wire, employable by the patient, adapted to enable the commanded changeover from the drawn pressure regime to the continuous pressure regime, from the positive pulsations regime to the negative pulsations regime, and vice versa.

8. Apparatus, according to claim 1, characterized in that the distribution of air can be complemented by the distribution of oxygen in the case of oxygen-dependent patients.
